SEQUENCE LISTING (1 sequence)

SEQ ID NO: 1 - protein, 216 aa, Artificial Sequence
  Description of Artificial Sequence Synthetic polypeptide
AVIKPDMKIKLRMEGSVNGHRFRIEGVGLGKPYEGKQSMDLKVKEGGPLPFAYDILTMAFCYGNRVFAKYPENIVDYFKQSFPEGYSWERQMIYEDGGICVATNDITLDGDCMISEIRFKGVNFPANGPVFQKRTVKWELSHEKLYARDGLLYSDGNYALSLEGGGHYRCDNKTTYKAKKVVQLPDYHSVTHHIVIKSHDKDYSNVNLHEHAEAHS

(19) United States
(12) Patent Application Publication
(10) Pub. No.: US 2023/0243838 A1
(43) Pub. Date: Aug. 3, 2023

(54) ENGINEERED FLUORESCENT SPONTANEOUS ISOMERIZATION RATE BIOSENSORS

(71) Applicant: Calico Life Sciences LLC, South San Francisco, CA (US)

(72) Inventors: Maria Ingaramo, South San Francisco, CA (US); Andrew York, South San Francisco, CA (US)

(21) Appl. No.: 17/904,089

(22) PCT Filed: Feb. 12, 2021
(86) PCT No.: PCT/US2021/017878

§ 371 Date: Aug. 11, 2022

Related U.S. Application Data
(60) Provisional application No. 62/976,947, filed on Feb. 14, 2020.

(51) Int. Cl. G01N 33/58; G01N 33/566

(52) U.S. Cl. CPC G01N 33/582; G01N 33/566

(57) ABSTRACT

Described herein are countdown biosensors, and methods of using the same, comprising fluorophores which can spontaneously photoswitch between two or more states with different fluorescent properties (e.g. fluorescent intensity or fluorescent color). The countdown sensor comprises a fluorescent domain which can spontaneously photoswitch, and a sensing domain which responds to the desired input. The countdown sensor is “read” by measuring the photoswitching rate. In certain embodiments, the decay of fluorescent intensity over time (due to spontaneous photoswitching of different fluorescent domains) can be made to depend on the concentration of different small molecules, such as calcium and nicotinamides.

### CROSS REFERENCE TO RELATED APPLICATIONS

This application is the U.S. National Stage entry of International Application No. PCT/US2021/017878, filed Feb. 12, 2021, which claims priority to U.S. Provisional Application No. 62/976,947, filed Feb. 14, 2020, both of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Jan. 31, 2023, is named CLS-022WOUS_SL.txt and is 2,346 bytes in size.

### BACKGROUND

Fluorescent tags (e.g., Alexa 488 or Green Fluorescent Protein) are often used to reveal the presence, distribution, and dynamics of large (multi-kilodalton) cellular components (e.g., proteins like actin or histones). Many molecules (e.g., glucose or calcium) are too small to be tagged via traditional fluorophores, and are sometimes referred to as the “dark matter” of fluorescence microscopy. “Small-molecule” concentrations and dynamics are just as interesting to biologists as “large-molecule” dynamics, but are often neglected, since they're so much harder to measure.

A subclass of fluorescent tags exists which reveals small molecule dynamics; we'll refer to them as “fluorescent biosensors”. For example, GCaMP combines a fluorescent domain (similar to GFP) with a calcium-binding domain (similar to calmodulin). The fluorescent intensity of a GCaMP molecule changes in response to the local concentration of calcium, revealing clues about calcium concentration and dynamics. There are many other examples of similar biosensors (e.g., Perceval or Peredox) which sense other small molecules (e.g., adenosine phosphates or nicotinamides).

Many fluorescent biosensors (which we'll call “intensity sensors”) suffer from an ambiguity: if they emit more (or less) photons, it could be due to changes in the concentration of their target, but it could also be due to changes in the concentration of the fluorescent biosensor (or changes in the instrument). For some questions (e.g., did this neuron fire?), this ambiguity is irrelevant. For other questions (e.g., is the pH in this yeast vacuole higher or lower today than it was yesterday?), this ambiguity is crippling.

A small subclass of fluorescent biosensors exists (which we refer to as “lifetime sensors”) which do not suffer from this ambiguity. Lifetime biosensors reveal changes in the concentration of their small-molecule target by changing their “fluorescent lifetime” (the number of nanoseconds that typically elapses between absorption and emission of light by the fluorescent domain). Since fluorescent lifetime does not depend on the concentration of the fluorescent biosensor (or aspects of the instrument), lifetime sensors enable long-term quantitative measurement of small molecule concentrations.

Unfortunately, measuring fluorescent lifetime is much slower, more complicated, and expensive vs. measuring fluorescent intensity, forcing biologists to choose between a fast, cheap, ambiguous measurement, and a robust, quantitative, slow, expensive measurement. It's also extremely difficult and laborious to construct novel fluorescent biosensors. Conceptually simple operations (e.g., changing the color of the fluorescent domain, or the target of the binding domain) typically require several years of mutation and screening, and often end in failure.

### SUMMARY

In certain aspects, described herein are protein biosensors, comprising a fluorescent domain; and an analyte binding domain; wherein the fluorescent domain can spontaneously photoswitch by cis-trans isomerization or protonation; and wherein the rate of isomerization or rate of protonation is altered by binding of the analyte binding domain to an analyte of interest. In certain embodiments, the photoswitching changes fluorescent intensity or the fluorescent color of the fluorescent domain. In certain embodiments, the analyte binding domain is attached to the N-terminus of the fluorescent domain. In certain embodiments, the analyte binding domain is attached to the C-terminus of the fluorescent domain. In certain embodiments, the fluorescent domain is green fluorescent protein or rsCherry. In certain embodiments, the analyte of interest is Calcium. In certain embodiments, the analyte of interest is nicotinamide. In certain embodiments, the protein biosensor comprises SEQ ID NO. 1.

In certain aspects, described herein are methods of making a protein biosensor, comprising attaching an analyte binding domain to a fluorescent domain; wherein the fluorescent domain can spontaneously photoswitch by cis-trans isomerization or protonation; and wherein the rate of isomerization or rate of protonation is altered by binding of the analyte binding domain to an analyte of interest. In certain embodiments, the protein biosensor comprises SEQ ID NO. 1.

In certain aspects, described herein are methods of identifying the concentration of an analyte of interest in a sample, comprising contacting the sample with a protein biosensor of any one of the above claims. In certain embodiments, the change in fluorescent intensity of the biosensor is correlated with the concentration of the analyte of interest in the sample. In certain embodiments, the change of fluorescent intensity of the biosensor is not dependent on the concentration of the biosensor. In certain embodiments, the protein biosensor comprises SEQ ID NO. 1.

In certain aspects, described herein are kits comprising the protein biosensor and instructions for use.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a diagram illustrating the improvements of countdown sensors compared to fluorescent intensity sensors and lifetime fluorescent sensors that leads to fast, simple and quantitative results. FIG. 1A is a diagram showing the structure of the countdown biosensor fluorophore before and after isomerization. FIG. 1B is a diagram showing the change in fluorescence and the structure of the countdown sensor fluorophore before and after isomerization. FIG. 1C is an image showing different quantitative intensities of the fluorescence of the sensors. FIG. 1D is a diagram showing the isomerization reaction of the countdown sensor.

FIG. 2 are graphs of the isomerization half-life and spontaneous deactivation half-life of a countdown sensor.

FIG. 3A shows an illustration of the structure of a calcium countdown sensor.

FIG. 3B is a graph showing the differences in spontaneous isomerization rate of a calcium countdown sensor when applied to solutions containing the shown concentrations of calcium.

FIG. 4A is a diagram illustrating the structure of a countdown sensor bound to a protein comprising a calcium binding domain at the N and C terminus of the fluorophore, leaving the fluorophore barrel structure intact.

FIG. 4B is a diagram of the structure of a protein calcium binding domain attached to the fluorophore outside of the N and C terminus where the barrel structure is opened.

FIG. 5A are graph showing the decrease in normalized fluorescence intensity before chelation of calcium with addition of EGTA, and after addition of EGTA where responsive calcium countdown sensors display a large reduction in fluorescence prior to EGTA compared to after addition of EGTA;

FIG. 5B are a series of graphs showing additional variants of calcium countdown biosensors, where a majority are responsive to calcium.

FIG. 6 is a graph showing the decrease in fluorescence intensity before and after addition of EGTA of a variant red fluorescent countdown sensor rationally designed by structural alignment to original green countdown sensor.

FIG. 7A is a diagram illustrating the structure of a nicotinamide countdown sensor comprising a glowing countdown domain (a fluorophore) attached to a Nicotinamide binding domain.

FIG. 7B are graphs showing the decrease in normalized fluorescence intensity of nicotinamide countdown sensors after addition of nicotinamide, where responsive nicotinamide countdown sensors display a large reduction in fluorescence after addition of NAD+ as compared to after addition of NADH; and series of graphs showing eight additional variants of nicotinamide countdown biosensors, where a majority are responsive to NAD+

### DETAILED DESCRIPTION

### Definitions

Terms used in the claims and specification are defined as set forth below unless otherwise specified.

As used herein, the term “biosensor” or “sensor” refers to a molecule that can detect a desired biological phenomenon or characteristic of interest.

As used herein, the term, “analyte binding domain” or “sensor domain” refers to a protein domain that can bind to an analyte (e.g., molecule or ion) of interest.

As used herein, the term “isomerization rate” or “photoswitching rate” refers to the rate of cis-trans isomerization or protonation of a fluorophore.

As used herein, the term “spontaneous isomerization rate sensors” or “countdown sensor” refers to a protein comprising the combination of a fluorescent domain which can spontaneously photoswitch, with a sensing domain which responds to a desired input (e.g., such as the concentration or availability of a molecule of interest).

As used herein, the term “photoswitch” refers to cis-trans isomerization or protonation of a fluorophore.

It must be noted that, as used in the specification and the appended claims, the singular forms “a,” “an” and “the” include plural referents unless the context clearly dictates otherwise.

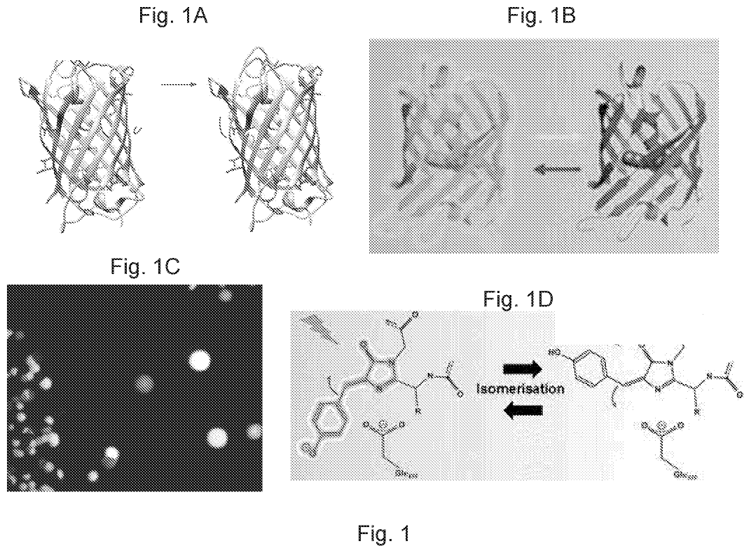

Described herein is a class of biosensors, spontaneous isomerization rate sensors, or “countdown sensors” that combine the ease-of-use of an intensity biosensor with the unambiguous quantitation of a lifetime biosensor (FIG. 1). Also described herein are novel engineered fluorescent domains (“countdown” domains) which are especially well suited to be used as the fluorescent domain of a countdown sensor. In addition, described herein are methods to modularly combine appropriate fluorescent domains with appropriate sensing domains in order to rapidly, easily produce novel countdown sensors.

Countdown Sensors

A subclass of fluorophores can spontaneously “photoswitch”: they change shape (e.g., via cis-trans isomerization or protonation) between two or more states with different fluorescent properties (e.g. fluorescent intensity or fluorescent color). A “countdown sensor” is the combination of a fluorescent domain which can spontaneously photoswitch, with a sensing domain which responds to the desired input (e.g. calcium concentration). The countdown sensor is “read” by measuring the photoswitching rate, and, for example, comparing the observed rate to a calibration curve of rate vs. quantity-to-be-sensed (e.g. calcium concentration). The decay of fluorescent intensity vs. time (due to spontaneous photoswitching of different fluorescent domains) can be made to depend on the concentration of different small molecules (in this case, calcium and nicotinamides).

Since spontaneous photoswitching rate does not depend on the concentration of the countdown sensor, or the measurement instrument, such measurements can be quantitative like a lifetime sensor. Since spontaneous photoswitching rates can be measured with a series of fluorescence intensity measurements, such measurements can be fast, cheap, and easy like an intensity sensor.

Unfortunately, the vast majority of fluorophores which spontaneously photoswitch do so extremely slowly (spontaneous photoswitching half-lives of hours or even days). Disclosed herein are a novel family engineered green fluorescent proteins which spontaneously photoswitch with half-lives ranging from hours to seconds. Their photoswitching rates become faster or slower depending on perturbations applied to the two “tails” of the protein (their C and N termini). For example, SEQ ID NO: 1 encodes of one of these engineered rapidly spontaneously photoswitching green fluorescent proteins.

This disclosure includes many other examples of spontaneously phostoswitching fluorescent proteins, which can vary in their spontaneous photoswitching half-life, their cross-section for light-driven on-switching, their cross-section for light-driven off-switching, their equilibrium degree of activation, the brightness/intensity of their “on” state, the brightness/intensity of their “off” state, their maturation time, and their tendency to oligomerize. Informally, the spontaneous photoswitching rate of the protein “countdown” sensor depends on how you tug on its tails (i.e., put strain on the N and/or C termini of the fluorescent domain). This is also true of other rapidly spontaneously photoswitching fluorescent proteins, for example, rsCherry.

Methods of Engineering Countdown Sensors

There are many ways to use this readout of “tugging” property to construct a biosensor. For example, a countdown sensor can be attached to a calcium binding domain to produce a green calcium countdown sensor. In another example, rsCherry can be attached to a calcium binding domain to produce a red calcium countdown sensor. In yet another example, rsCherry can be attached to a nicotinamide binding domain to produce a red nicotinamide sensor.

Construction of novel biosensors including the above disclosed examples are engineered and constructed rapidly due to the remarkable modularity of countdown sensors. By simply attaching selected binding domains for an analyte of interest to the native C and N termini of a spontaneously photoswitching fluorescent domain, the fluorescent properties are preserved to an exceptional degree, primarily modulating their photoswitching rates with minimal effects on other properties like brightness.

Note that countdown sensors described herein can be used beyond sensing small molecule binding via coupling to a binding domain. Any process that can be made to “tug” on the “tails” of a rapidly spontaneously photoswitching fluorescent protein can be measured via the same optical measurement. For example, “countdown” sensors could be used to measure cellular physiological phenomenon, such as, but not limited to, being used as a tension sensor for actin cytoskeletal dynamics by coupling it to components of the cytoskeleton.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1: Construction of Calcium Responsive Countdown Sensor

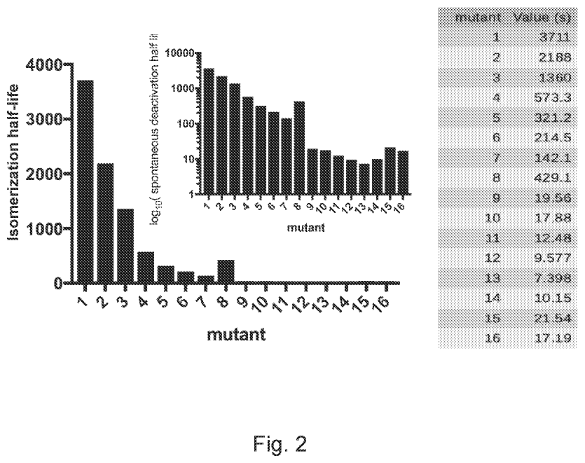

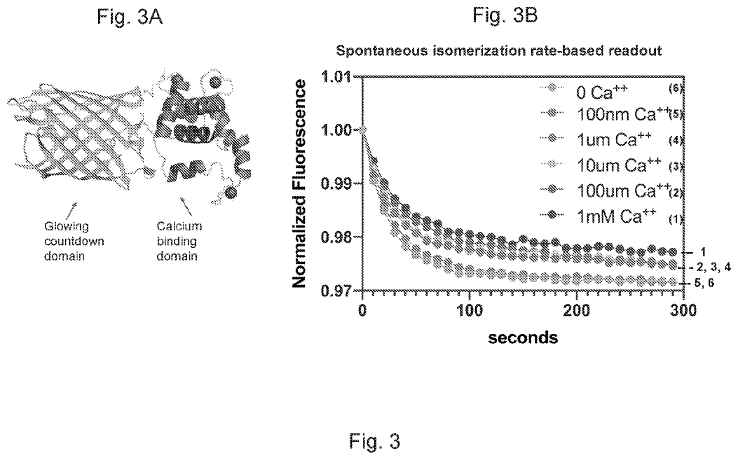

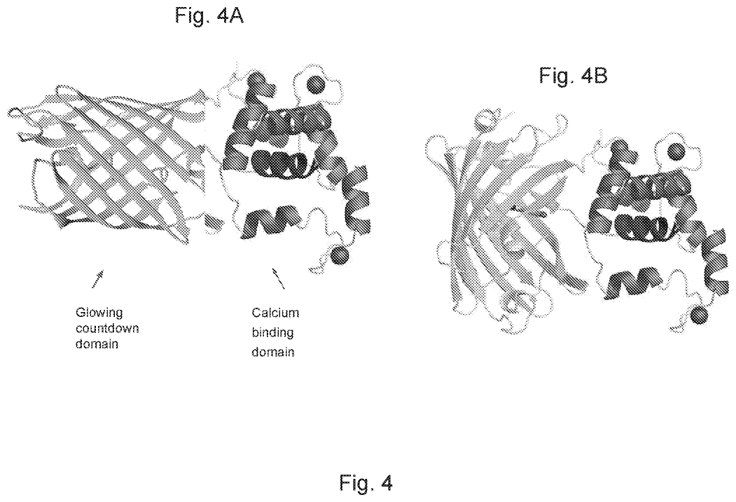

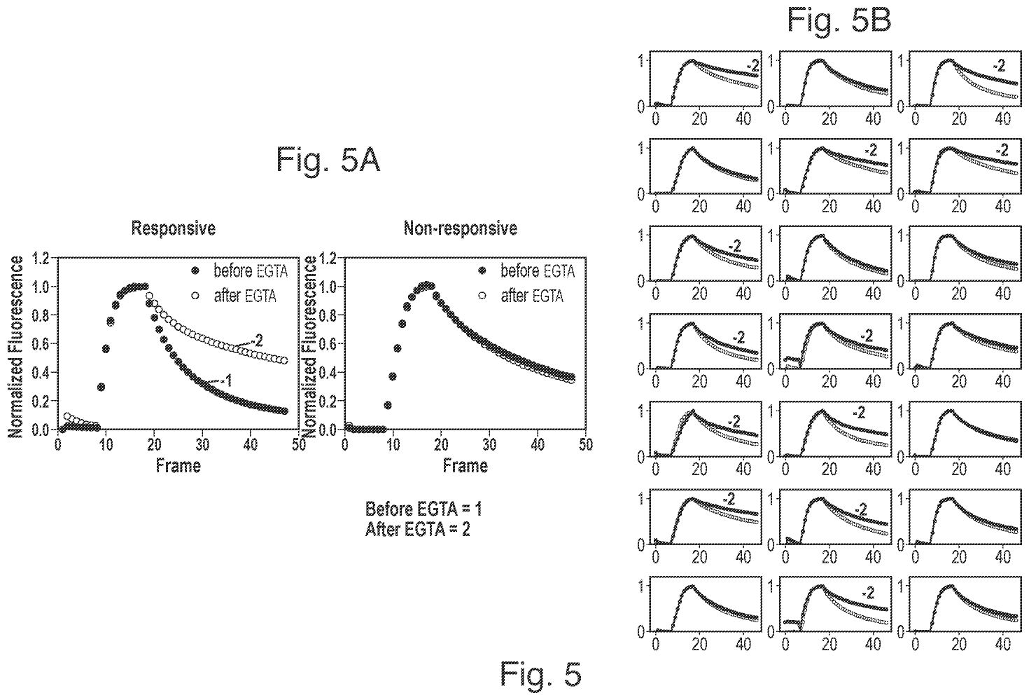

A green fluorescent calcium countdown sensor was constructed by fusing a calcium binding domain to a green fluorescent protein (GFP) domain at the N or C terminus (FIGS. 1-5). The green calcium countdown sensor was placed in solutions with either no Calcium, or 100 nm, 10 uM, 100 uM, or 1 mM Ca²⁺, and the fluorescence intensity was measured over 300 seconds (FIG. 3). The decrease in fluorescence intensity, and thus, spontaneous isomerization rate correlated quantitatively with the corresponding concentration of calcium. To screen for additional green fluorescent calcium countdown sensor variants responsiveness to calcium, the green fluorescent calcium countdown sensors were also placed in a solution of calcium and fluorescence intensity was measured over time before and after addition of EGTA (FIG. 5). Using this screen, a majority of the variant green fluorescent calcium countdown sensors were found to be responsive to calcium.

### Example 2: Construction of Calcium Responsive Countdown Sensor with Different Color Fluorescence

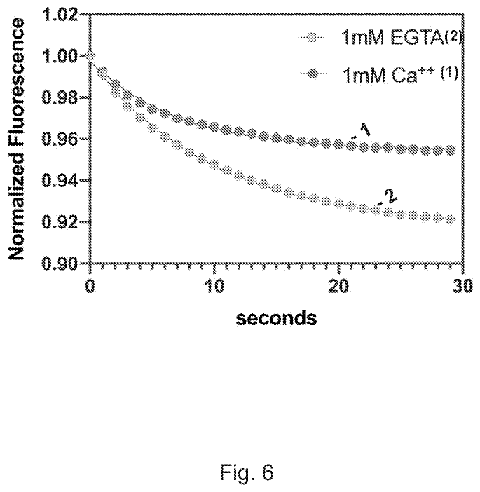

A red fluorescent calcium countdown sensor was constructed by fusing a calcium binding domain to an rsCherry fluorescent protein domain at the N or C terminus (FIG. 6). The rsCherry domain was designed by aligning its structure with the green countdown sensor structure. The fluorescence of the red fluorescent calcium countdown sensor was measured when placed in a solution containing 1 mM Ca²⁺, before and after addition of EGTA (FIG. 6). The fluorescence decreased significantly after addition of EGTA; thus, the red fluorescent calcium countdown sensor is responsive to calcium.

### Example 3: Construction of Nicotinamide Responsive Countdown Sensor

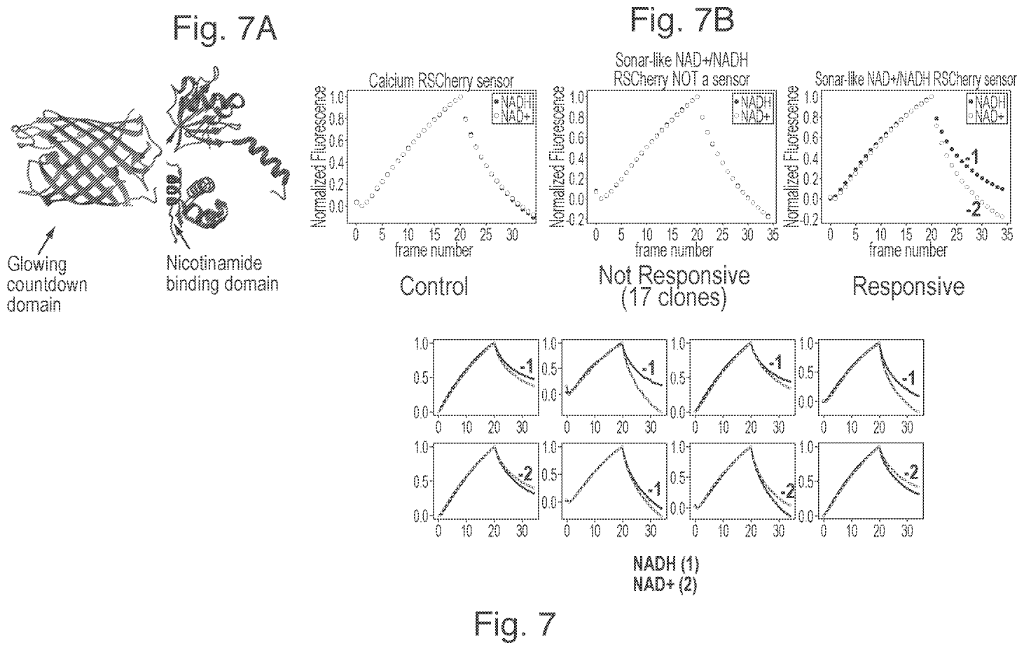

A fluorescent nicotinamide countdown sensor was constructed by fusing a nicotinamide binding domain to a fluorescent protein countdown domain at the N or C terminus (FIG. 7). The fluorescence of the fluorescent nicotinamide countdown sensor was measured when placed in a solution containing NAD+ or NADH. The fluorescence decreased significantly after addition of NAD+; thus, the nicotinamide countdown sensor is responsive to oxidized nicotinamide concentrations.

### Example 4: Construction and Optimization of Countdown Sensors

Additional countdown sensors are constructed to respond to small molecules or ions and other small targets of interest. Analyte binding domains are fused to different fluorescent countdown sensors of different fluorescent colors and intensities and the countdown sensor is optimized by testing for a reduction in fluorescent intensity (or change in fluorescent color) upon reduction in the target concentration or availability.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### Informal Sequence Listing

SEQ ID NO. 1
AVIKPDMKIKLRMEGSVNGHRFRIEGVGLGKPYEGKQSMDLKVKEGGPL
PFAYDILTMAFCYGNRVFAKYPENIVDYFKQSFPEGYSWERQMIYEDGG
ICVATNDITLDGDCMISEIRFKGVNFPANGPVFQKRTVKWELSHEKLYA
RDGLLYSDGNYALSLEGGGHYRCDNKTTYKAKKVVQLPDYHSVTHHIVI
KSHDKDYSNVNLHEHAEAHS

1. A protein biosensor, comprising:
a fluorescent domain; and
an analyte binding domain; wherein
the fluorescent domain can spontaneously photoswitch by cis-trans isomerization or protonation; and wherein
the rate of isomerization or rate of protonation is altered by binding of the analyte binding domain to an analyte of interest.

2. The protein biosensor of claim 1, wherein the photoswitching changes fluorescent intensity or the fluorescent color of the fluorescent domain.

3. The protein biosensor of claim 1 or 2, wherein the analyte binding domain is attached to the N-terminus of the fluorescent domain.

4. The protein biosensor of claim 1 or 2, wherein the analyte binding domain is attached to the C-terminus of the fluorescent domain.

5. The protein biosensor of any one of claims 1-4, wherein the fluorescent domain is green fluorescent protein (GFP) or rsCherry.

6. The protein biosensor of any one of the above claims, wherein the analyte of interest is Calcium.

7. The protein biosensor of any one of the above claims, wherein the analyte of interest is nicotinamide.

8. A method of making a protein biosensor, comprising:
attaching an analyte binding domain to a fluorescent domain; wherein
the fluorescent domain can spontaneously photoswitch by cis-trans isomerization or protonation; and wherein
the rate of isomerization or rate of protonation is altered by binding of the analyte binding domain to an analyte of interest.

9. A method of identifying the concentration of an analyte of interest in a sample, comprising contacting the sample with a protein biosensor of any one of the above claims.

10. The method of claim 9, wherein
the change in fluorescent intensity of the biosensor is correlated with the concentration of the analyte of interest in the sample.

11. The method of claim 9 or 10, wherein
the change of fluorescent intensity of the biosensor is not dependent on the concentration of the biosensor.

12. The protein biosensor of any one of the above claims, wherein the protein biosensor comprises SEQ ID NO. 1.

13. A kit comprising the protein biosensor of any one of the above claims and instructions for use.
